(19) United States
(12) Patent Application Publication
(10) Pub. No.: US 2004/0147818 A1
(43) Pub. Date: Jul. 29, 2004

(54) Portable system for monitoring and processing patient parameters in multiple oprational modes

(76) Inventors: Andrew Levy, Charlestown, MA (US); Wolfgang Scholz, Beverly, MA (US); Tomas Russ, Carlisle, MA (US); Clifford M. Kelly, Windham, NH (US); Joseph Elaz, North Andover, MA (US); Kenneth Fuchs, Wayland, MA (US)

Correspondence Address: Jack Schwartz & Associates, Suite 1507, 1350 Broadway, New York, NY, 10018, US

(21) Appl. No.: 10/712,758

(22) Filed: Nov. 12, 2003

Related U.S. Application Data
(60) Provisional application No. 60/427,154, filed on Nov. 18, 2002.

(51) Int. Cl. A61B 5/00

(52) U.S. Cl. 600/300; 340/539.12

(57) ABSTRACT

A system, housed as a portable monitoring unit, monitors and processes signal parameters acquired from a patient in multiple operational modes. A data acquisition processor receives patient parameter data from a plurality of different patient attached sensors and processes that data to provide processed patient parameter data. An image reproduction device displays processed patient parameter data. A communication interface communicates the processed patient parameter data to the image reproduction device for display and concurrently communicates the processed patient parameter data to either a docking station when said portable monitoring unit is docked in said docking station in a first mode or a network access point coupled to a communication network via wireless communication in a second mode. A power unit re-charges a battery in the portable monitoring unit in the first mode.

The present patent application claims priority from provisional patent application No. 60/427,154 filed on Nov. 18, 2002 by C. M. Kelly et al.

### BACKGROUND OF THE INVENTION

1. Field of the Invention

The present invention relates generally to the field of medical devices. More particularly, the present invention relates to a monitoring device which can be utilized in many different operational modes.

2. Background of the Invention

Physiological monitoring devices as they exist today are typically designed for the specific locales in which they are used when rendering care to a patient, e.g. inside or outside of a hospital. In the case of a trauma patient, the monitoring of the patient will typically begin at the site of an accident. Ambulances carry support transport monitors which have been designed to be rugged so that they can be used in mobile ground and air vehicles.

Continuing the example, the patient is transported to the hospital and may originally be admitted to the emergency department for evaluation of the severity of the accident or illness. This usually requires disconnecting the patient from the ambulance monitor and reconnecting the patient to the monitor in the emergency room (ER). Monitors for the emergency room area of a hospital will often have a wireless connection to a central station and information network which allows the monitor and the patient to be mobile. In this manner they can be deployed, viewed and controlled where needed in the ER.

If the patient is critically ill, the patient may be taken to an operating room (OR) or to the intensive care unit (ICU). If this occurs the patient may be disconnected from the ER monitor and reconnected to the OR or ICU monitor. The monitors utilized in the OR and ICU typically are stationary and fixed to the wall. They also may provide larger and higher resolution displays to allow easy access to a large amount of data. The monitors in the OR or the ICU may also allow more parameter functions to be added to get a more complete picture of the health of the patient.

The problem in monitoring the patient continues when the patient is moved from the OR to a recovery area and then to an ICU area. From the ICU area the patient may be moved to a less critical “step-down” unit. In these instances the patient may again have to be physically disconnected from the OR monitor and reconnected to a transport monitor and then to the ICU or step-down monitor.

It is also sometimes desirable to outfit healthier patients with wearable (telemetry) devices that are semi-mobile or mobile to allow the patient to leave the bedside such as to go to the toilet and/or to ambulate within the care unit. Some patients who are fully ambulatory may be permitted to exercise by walking within a specific area of the hospital. The monitor for this use needs to be small and lightweight so that the patient may easily carry it while walking or exercising. After the patient further improves in physiological status, the patient may no longer need to be continuously monitored. However the vital signs are periodically spot-checked so that the patient can eventually be given a final complete evaluation before discharge.

Current patient monitoring devices also may be fabricated in different sizes due to differences in patient sizes. For example, small monitors may be required for neo-natal patients, slightly larger monitors for pediatric patients and full size monitors for adult patients. Such monitors also include different monitoring parameters for the different patient categories. For example, infant heart rates are higher than adult heart rates, and alarm parameters need to be set appropriately.

As described above, it is possible for hospitals to have two to four or even more different types of monitor devices to cover the care locales, applications and patient categories, as described above. Because these types of monitoring devices are not necessarily manufactured by the same vendor they are unlikely to use identical communication protocols and methods and will most likely have different controls, interfaces and/or accessories. Having multiple monitors, therefore, requires extra work from the nursing staff. They have to connect and disconnect the sensors attached to the body of the patient whenever a monitor is exchanged. Also even though accessories are only used occasionally it is cumbersome to disconnect an accessory for one monitor from the patient and then to reconnect the corresponding accessory for a different monitor. Furthermore, the nursing staff has to be trained to operate the multiple monitoring devices with their different controls and interfaces.

Another problem is that prior systems lack multiple device intercompatibility. That is, different devices do not easily communicate with one another, or do not communicate in any manner. Consequently, it is difficult or impossible to transfer data from one type of monitoring device to another. For that reason, history data for the patient may be lost as the patient progresses through the different areas of the hospital. Further, because the patient is sometimes disconnected from any monitoring device a complete record of physiological data for that patient is impossible to maintain.

Some existing monitoring devices have attempted to combine transport monitor features with fixed monitor features to avoid interrupting collection of data. More specifically, such devices attempt to combine large displays with portability. In such an arrangement a compromise is generally required. If the display is made large enough for high resolution use then the unit becomes too bulky and heavy for transport. If the display is designed to be lightweight and compact in size for portability the monitor does not have an adequate screen size and resolution and accessing the data may be difficult or not possible. Other existing monitors lack a local display, delaying immediate assessment of a patient condition and are costly to purchase and maintain.

### BRIEF SUMMARY OF THE INVENTION

The inventors have advantageously recognized the need for a monitor that may reduce or eliminate the requirement that patients be disconnected from one monitor and reconnected to another monitor as they pass through different locales and different stages of their recover from initial emergency transport to ambulatory outpatient; one that may adopt its operation automatically to the needs of the patient and the patient's environment; and also one that is a compact, hand held device that nurses can carry from patient to patient as required.

In accordance with principles of the present invention, a system, housed as a portable monitoring unit, monitors and processes signal parameters acquired from a patient in multiple operational modes. A data acquisition processor receives patient parameter data from a plurality of different patient attached sensors and processes that data to provide processed patient parameter data. An image reproduction device displays processed patient parameter data. A communication interface communicates the processed patient parameter data to the image reproduction device for display and concurrently communicates the processed patient parameter data to either a docking station when said portable monitoring unit is docked in said docking station in a first mode or a network access point coupled to a communication network via wireless communication in a second mode. A power unit re-charges a battery in the portable monitoring unit in the first mode.

### BRIEF DESCRIPTION OF THE DRAWING

FIG. 1a is a block diagram of a portable monitoring unit used in a system incorporating the principles of the present invention;

FIG. b is a three-dimensional view of the external housing which forms the portable monitoring unit of FIG. 1a;

FIG. 2 is a block diagram illustrating the portable monitoring unit of FIG. 1a arranged in a docking station which may be connected to a communication network;

FIG. 3 is a block diagram illustrating the portable monitoring unit of FIG. 1 connected to a wireless transceiver for communication with a network;

FIG. 4 is a block diagram in which the portable monitoring unit serves as a wired front end of a clinical workstation;

FIG. 5 is a block diagram illustrating the portable monitoring unit of FIG. 1a operating as a wireless front end of a clinical workstation; and

FIG. 6 is a block diagram of the portable monitoring unit of FIG. 1a equipped with a standard “Compact Flash” slot for use with a variety of Compact Flash expansion pods.

### DETAILED DESCRIPTION OF THE INVENTION

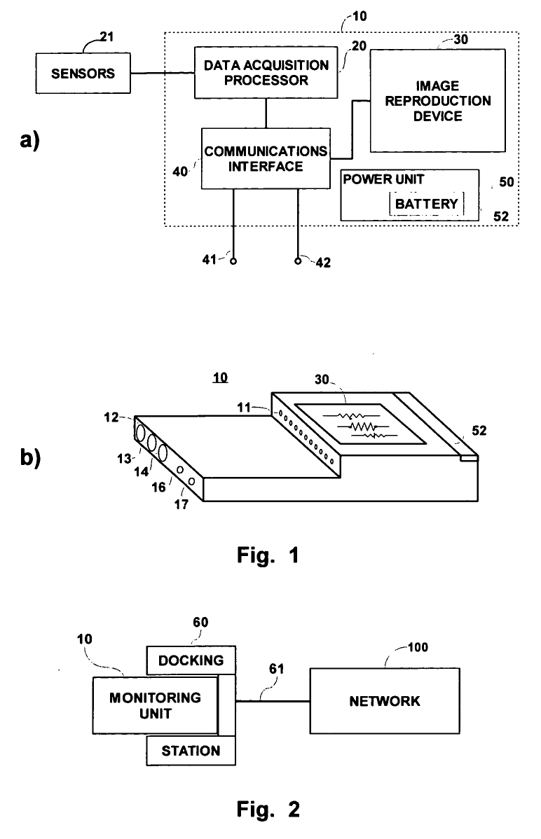

Referring to the drawings and more particularly to FIG. 1a, a system for monitoring and processing patient physiological parameters includes a portable monitoring unit 10. The monitoring unit 10 contains a data acquisition processor 20, an image reproduction device 30, a communications interface 40 and a power unit 50 including a battery 52. The communications interface 40 permits the portable monitoring unit 10 to be operated in a plurality of operational modes. The communications interface 40 is shown to have terminals, 41 and 42, which are available for connection to external sources. While only two terminals are shown it should be appreciated that more than two terminals may be available from the portable monitoring unit 10. The power unit 50 is connected to the other elements of the portable monitoring unit 10 but such connections are not shown in order to avoid unnecessarily complicating the drawing.

Sensors 21 are illustrated connected to the data acquisition processor 20. The sensors 21 are not necessarily a part of, or mounted on, the portable monitoring unit 10 but are available for attachment to a patient for transmitting signals corresponding to physiological functions from the patient to the data processor 20. Such physiological data may include (a) electrocardiograph (ECG) data, (b) blood parameter data, (c) ventilation parameter data, (d) infusion pump related data, (e) invasive or non-invasive blood pressure data, (f) pulse rate data, (g) temperature data and/or (h) respiratory data. For example, sensors 21 may be ECG electrodes which may be attached to the chest of a patient and connected via a wiring harness to the data acquisition processor 20 of the portable monitoring unit 10.

Terminals 41 and 42 communicate with external systems. The communications interface 40 transmits data representing patient physiological parameters to these external systems on one or more of the terminals 41 and 42 as described in more detail below.

The monitoring unit 10 is a miniaturized, lightweight monitoring device, which can measure essential physiological parameters of a person connected to the sensors 21. The monitoring unit 10 as shown in FIG. 1a has both wired and wireless capabilities for self-contained use in mobile areas such as field transport, emergency room and step-down/telemetry. As will be shown, the monitoring unit 10 may also be seamlessly interfaced with a clinical workstation in a hospital, for example, as an intelligent front end for more advanced monitoring.

Referring to FIG. 1b, a three dimensional view is shown of the external housing which forms the portable monitoring unit 10 shown in FIG. 1a. The monitoring unit 10 contains a plurality of terminals which connect to the sensors 21 (not shown in FIG. 1b). For example, terminals 11 may be used for ECG measurements. In the embodiment illustrated in FIG. 1b, there are ten terminals 11 which may be used to connect to the ten electrodes which generate a standard twelve lead electrocardiogram. These terminals may be fabricated to receive a standard connector from an ECG electrode cable harness. A terminal 12 may be used to connect to a non-invasive sensor utilized for determining blood pressure and a terminal 13 may be used to connect to a sensor utilized for measuring blood oxygen levels (SpO₂). A terminal 14 may be used to connect to a sensor for measuring one or more temperatures at different anatomical areas of a patient. Terminals 16 and 17 may be used to connect to external devices to provide synchronization with those external devices, if necessary.

At the rear end of the monitoring unit 10 (not visible in FIG. 1b) are docking station connectors. These connections include terminals 41 and 42, used, as described above with reference to FIG. 1a, to communicate patient physiological parameter data to external systems. The battery 52 in the power unit 50 is also connected at the rear end of the monitoring unit 10 so that it can be charged when the monitoring unit 10 is connected to a docking station. The operation of the monitoring unit 10 when placed in a docking station will be described in detail below.

An image-reproducing device 30 is mounted in viewable position on the top surface of the housing of the monitoring unit 10. Referring to FIG. 1a, processed patient physiological parameter data is routed from the data acquisition processor 20 to the image reproduction device 30 by the communications interface 40. The image reproduction device 30 generates an image representing the received patient physiological parameter data. For example, ECG data may be displayed in the form of known real time ECG graphs for one or more leads. SpO₂ data may be displayed in textual form as a numerical percentage. Images representing other patient physiological parameter data may be displayed in appropriate form on the image reproduction device 30. In the illustrated embodiment, the image reproduction device 30 is a liquid crystal display (LCD) optimized in size and resolution for most field and in-hospital transport as well as for other clinical applications. The LCD display 30 may also include a swivel mount device, similar to a rear view mirror, which allows the LCD display 30 of the monitoring unit 10 to be aimed for viewing at a desired angle.

Referring now to FIG. 2, the monitoring unit 10 is shown inserted into a docking station 60. The docking station 60 contains connectors which correspond to the connectors on the monitoring unit 10. When the monitoring unit 10 is inserted into the docking station 60, the corresponding connectors in the monitoring unit 10 and the docking station 60 mate so signals may pass between them, in a known manner. The docking station 60, in turn, is connected to a network 100 via a connection 61. The connection 61 may be a cable containing one or more conductors or a wireless link. The network 100 may include a connection to other electronic equipment, another computer, a local area network (LAN), or a wide area network (WAN) which may include the internet as a component.

When the monitoring unit 10 is to be worn by an ambulatory patient the docking station 60 may be configured to resemble a holster which may be strapped to the patient or hung from a gurney on which the patient is transported. In this mode of operation, the connection 61 may be implemented as a wireless link. When the patient is at a fixed location, such as in a bed, a treatment or a therapy room, or on an operating table, the docking station 60 may be located at a fixed location near the patient wearing the monitoring unit 10. In this mode of operation, the docking station 60 may be connected to the hospital network 100 via a wired connection 61. Also in this mode of operation, a power connection may be made from the power mains in the hospital building to the battery 52 in the power unit 50 (FIG. 1), allowing the battery to recharge. In either of these operational modes, the connection 61 allows data representing the physiological parameter signals received from the sensors 21 (of FIG. 1a) attached to the patient's body to be transmitted from the monitoring unit 10 to the network 100 via the docking station 60.

For example, when a patient leaves a fixed location, the monitoring unit 10 may be removed from the fixed docking station 60 and placed in a holster 60. When a patient arrives at a fixed location, the monitoring unit 10 may be removed from the holster 60 and placed in a fixed docking station 60. Patient physiological parameter data, therefore, remains available to the network 100 whenever a patient is moved without having to physically remove and replace sets of sensors from the patient, either by physically removing and replacing the sensors, or by physically disconnecting and reconnecting any standard cable or connector connected to a set of sensors, as for ECG sensors.

The network 100 may include a larger image display device having a higher resolution to enable clinicians to read images representing patient physiological parameters with a higher degree of accuracy than is available from the smaller lower resolution image reproduction device 30 (FIG. 1) of the monitoring unit 10. The monitoring unit 10 may sense or query the network 100 to determine if a higher resolution display device is present on the network 100. Alternatively, the higher resolution display device may signal its presence on the network 100 to the monitoring unit 10. In either of these cases, the monitoring unit 10 automatically changes its operational mode to send the physiological parameter data to the higher resolution display device. The monitoring device 10 may also be conditioned manually to send the physiological parameter data to a higher resolution display device over the network 100. In either case, the monitoring unit 10 acts as a data processing front-end collecting signals from the sensors 21 and processing those signals to generate processed patient physiological parameter data. The processed patient physiological parameter data is routed over the network 100 to the large high-resolution display device. The large high-resolution display device displays images representing the received patient physiological parameter data.

Some hospitals have wired and/or wireless communication networks which permit a person at a central monitoring station, possibly including one or more large high-resolution image display devices, to monitor the physiological parameters of one or more patients. Such a central monitoring station may also be connected to the network 100. The patient physiological parameter data from the monitoring unit 10 may be routed via the network 100 to the central monitoring station, where it may be displayed and monitored. There may also be a central history data repository where a complete history of physiological parameter data is stored. The central history data repository is also connected to the network 100 and stores the physiological parameter data received from the monitoring unit 10.

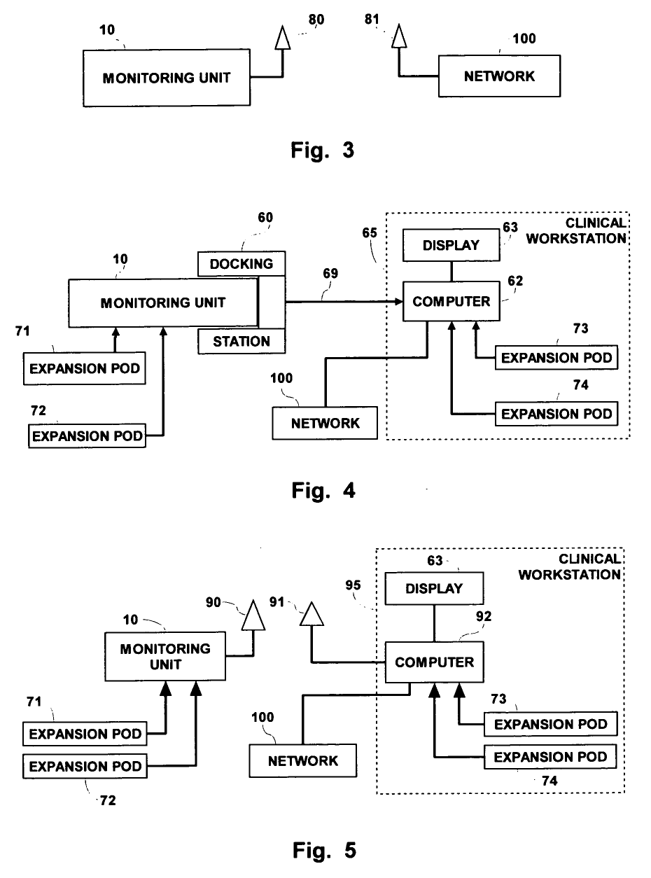

Referring to FIG. 3, the monitoring unit 10 is shown including a wireless network transceiver represented by antenna 80 which can communicate with a wireless access point 81. The wireless network transceiver 80 is connected to the communications interface 40 (FIG. 1) in a similar manner to terminals 41 and 42. The communications interface 40 supplies patient physiological parameter data from the data acquisition processor 20 to the transceiver 80. The wireless access point or other wireless transceiver represented by antenna 81 is connected to the network 100. The transceivers 80 and 81 may be Bluetooth 802.15 wireless transceivers. The Bluetooth technology is described for example in “Haartsen, Bluetooth, the Universal Radio Interface for Adhoc, Wireless Connectivity”, Ericsson Review #3, 1998, pages 110-117. The monitoring unit 10 may also include transceivers capable of network or local communication using other wireless technologies including WLAN 802.11b, 802.11a, 802.11g, Bluetooth 802.15 and GSM/GPRS.

Because this operational mode is available, the monitoring unit 10 is able to communicate with the network 100 whether docked in a docking station 60 (FIG. 2) or not (FIG. 3) while it remains connected to the patient via the sensors 21 (FIG. 1) which were initially applied to the patient's body. That it, is not necessary for sensors 21 to be removed from and replaced on the patient, nor for any cable connector attached to sensors (e.g. ECG cable connector) to be disconnected from and reconnected to a monitoring unit 10, when moving from one location to another. The communications interface 40 (FIG. 1) is able to automatically detect when a wired connection to the network 100 is not available, and to automatically enable the wireless transceiver 80 and supply the patient physiological parameter data to the network 100 via the wireless transceiver 80 instead.

In FIG. 3, the monitoring unit 10 may operate in a wearable telemetry mode. In this operational mode, the image reproduction device 30 (FIG. 1) is switched off most of the time to conserve power. More specifically, the image reproduction device 30 may be switched off after a predetermined time interval to conserve power in response to a preprogrammed instruction. The battery life, thus, may be extended from several hours to a few days. The nurse may use the display intermittently during rounds to check on the patient's condition or to adjust sensor or electrode placement when the nurse is at the bedside of the patient. This arrangement is an advantage over present telemetry systems which require the nursing staff to either go to a remote location at a central station to view the physiological parameters of the patient or to obtain and carry a secondary receiving device to view data from the telemetry transmitter.

Some large high-resolution display devices, described above, also include more functions for analyzing the received physiological parameter data. Such devices are referred to as clinical workstations. Such a workstation 65 is shown in FIG. 4. In FIG. 4 the monitoring unit 10 is docked into the docking station 60 which is connected to a computer 62 within the clinical workstation 65 via an interface connection 69. The computer 62 is connected to a larger higher resolution display 63 within the clinical workstation 65 and to the network 100.

When the monitoring unit 10 is connected directly to the network 100, either through a docking station 60 (FIG. 2) or through a wireless connection (FIG. 3), the monitoring unit 10 senses the presence of a network connection by any appropriate method. For example, the monitoring unit 10 may query for a network address. If one is received, the monitoring unit 10 determines that it is connected to the network 100 and automatically enters the operating mode for a direct connection to the network 100.

When the monitoring unit 10 is physically docked into a docking station 60 that is connected to the computer 62 in the clinical workstation 65, the monitoring unit 10 senses the presence of a workstation connection by any appropriate method. For example, the monitoring unit 10 may query for a network address. In this case, however, the computer 62 returns a signal to indicate that the monitoring unit 10 is connected to a clinical workstation 65 and not to the network 100. More specifically, it may return a specific network address which is reserved to indicate a connection to a clinical workstation 65, or may return any other signal suitable for indicating that the monitoring unit 10 is not connected to the network 100. In response, the monitoring unit 10 determines that it is connected to a clinical workstation 65, and automatically changes its operating mode for a connection to a clinical workstation 65.

In this operating mode, the monitoring unit 10 operates as an intelligent front-end module that is one component of a larger point-of-care system. The monitoring unit 10 receives signals representing patient physiological parameters from the sensors 21 (FIG. 1), processes those signals, and communicates the processed physiological parameter data to the clinical workstation 65. The clinical workstation 65 has a large display screen 63 and extended applications, controlled by a computer 62, to form a single complete high-end patient monitor. This point-of-care system is connected to the network 100 and may provide data to a central monitoring station or patient history repository. Using the monitoring unit 10 as a data processing front-end for a clinical workstation allows the clinicians to use the larger higher resolution display of the physiological parameters along with any available additional functions, without requiring the set of sensors 21 (FIG. 1) to be removed from and replaced on the patient, or disconnecting and reconnecting any sensor cable connector.

The computer 62 may also be connected to expansion pods 73 and 74 respectively. These expansion pods 73, 74 are easily added or removed, and provide additional capabilities, described below, to the clinical workstation 65. The expansion pods 73, 74 may be fabricated in known small insertable packages such as PCMCIA, Compact Flash or other insertable packages, or may be attachable via a wired or wireless connection using known communications protocols such as USB or Fire Wire. In either case the computer 62 includes corresponding terminals to which the expansion pods may be inserted or connected.

It is also possible for the monitoring unit 10 to include expansion pods, illustrated as expansion pods 71 and 72 in FIG. 4. In the case of the monitoring unit 10, its small size work make expansion pods implemented as insertable packages (e.g. PCMCIA or Compact Flash) more practical, though connectable expansion pods (e.g. USB or Fire Wire) are possible. The monitoring unit 10 may include terminals to which the expansion pods may be inserted or connected. Expansion pods 71, 72 in the monitoring unit 10 may be used for the same purposes described above for the computer 62 in the clinical workstation 65.

In FIG. 5, another embodiment is shown in which the monitoring unit 10 is connected to a wireless transceiver 90 (as in FIG. 3), which transmits data to a wireless transceiver or wireless access point 91. The wireless transceiver 91 is, in turn, connected to a computer 92 connected to a larger higher resolution display monitor 63. Both the monitoring unit 10 and the computer 92 can be connected to expansion pods 71, 72, 73 and 74 respectively, similar to the arrangement shown in FIG. 4. The computer 92 is then connected to a network 100. In FIG. 5 the monitoring unit 10 is wirelessly connected to a clinical workstation 95. Except for delivering processed patient physiological parameter data to the clinical workstation 95 wirelessly, the operation of the embodiment illustrated in FIG. 5 is the same as that illustrated in FIG. 4.

As noted above with respect to FIGS. 4 and 5, the monitoring unit 10 may be used as a “front-end” for the local clinical workstation computer to form a high-end monitor. The local “point-of-care” connection between the monitoring unit 10 and the display 63, both wired and wireless, advantageously employs special low latency protocol (LLP) requirements to ensure an extremely short delay between acquisition of the signals from the sensors 21 (FIG. 1) of the front-end acquisition unit (monitoring unit 10) and display of images representing those signals on the workstation 65 large screen 63. The low latency protocol allows the information on the large screen 63 to be used for time-sensitive applications such as hand to eye coordination by a surgeon when a catheter is placed in the body and the catheter is moved between heartbeats.

The low latency wireless connection (FIG. 5) also allows the monitoring device to have short-range mobility. This is desirable for example in the operating room when moving the patient into the OR and then preparing the patient for surgery. During this time period the physiological parameter data from the monitoring unit 10 (in front-end operational mode) may be wirelessly transmitted to the fixed computer 92 for display on the large screen 63 until the placement of the patient is completed and the monitoring unit 10 may be mounted in the fixed docking station 60 (FIG. 4).

The wireless connection may also be used to communicate locally with a mobile tablet computer in situations where the high-performance clinical workstation computer is not necessary but a screen larger than that in the monitoring unit 10 is desired. Such an arrangement would be the same as illustrated in FIGS. 4 and 5, except the processor 62 (or 92 in a wireless mobile tablet computer) is programmed to operate as a general purpose processor instead of being programmed to operate as the controller for a clinical workstation. The combination of the tablet computer and monitoring unit 10 is useful for applications such as medium or high-level transport. The fixed computer or tablet computer may also concurrently acquire additional information from other front-end devices. The monitoring unit 10 may also include additional local positioning radios for asset management, patient location and theft deterrence.

When wired network connections are being utilized, a standard such as Ethernet may be used as a private high-speed deterministic point-to-point network. It may be used in a traditional monitoring network with dozens of peers on the same network. In a wireless solution for these network connections, multiple radios can be used to optimize the data transmission rate and minimize the power consumption.

When the patient acuity reaches a certain level, the non-invasive sensors 21 (FIG. 1) built into the monitoring unit 10 may not be adequate to monitor the patient. In this case external pods 71, 72 shown in FIGS. 4 and 5 may be connected to the monitoring unit 10 to extend its functionality. The expansion pods 71, 72 may be connected to other, possibly more invasive, sensors or other medical equipment for gathering additional physiological parameter signals. For example, in intensive care situations, the necessity for highly accurate information may require in-situ measurements. Such sensors are generally classified as minimally-invasive or invasive. An example of minimally-invasive sensors for measuring blood perfusion is a needle probe inserted into a location of interest, such as a blood vessel, the brain, gingiva, or oral/nasal mucosa. An example of an invasive sensor is one which may be surgically implanted (some of which are adapted for chronic implantation) in the area of interest. In either case, the sensor generates a signal representing the level of the blood perfusion and is connected to an expansion pod 71, 72 which provides data representing the blood perfusion the monitoring unit 10.

Wireless physiological sensors also exist which may be implanted in an area for which accurate pressure measurements are desired. Such sensors may produce signals representing internal blood pressure, intracranial, gastrological and intrauterine pressure, urodynamic measurements by catherization, and other remote pressure readings. Such a sensor may be surgically implanted in the anatomical area of interest. In operation, the sensor transmits a wireless signal representing the pressure in that area. A corresponding expansion pod 71, 72 receives this signal and provides data to the monitoring unit 10 representing the sensed pressure.

Other invasive sensors exist for measuring flow of a liquid through a vessel, such as measuring blood flow through a blood vessel. Still other sensors exist which provide simultaneous parameters which represent tissue or organ vitality in real-time. All operate in-situ and send information to a corresponding expansion pod 71, 72, which in turn provides data to the monitoring unit 10 representing the sensed physiological parameter.

The monitoring unit 10 is designed to support these additional pods when docked in the holster or docking station 60 and also during wireless operation such as transport on battery power. Other expansion pods 73, 74 for providing access to more physiological data which may not be required during patient transport may be interfaced by connection to the clinical workstation computer 62 (or 92) as additional front-ends in parallel with the monitoring unit 10. This arrangement is also shown in FIGS. 4 and 5. The computer 62 (or 92) in the clinical workstation 65 (or 95) causes data representing the additional patient physiological data gathered by the expansion pods 71, 72, 73, and 74, respectively, to be displayed in an appropriate format on the large display 63 when requested by the clinician.

The monitoring unit 10 described above is small and easily carried. Therefore, it may be used as a hand-held vital signs “spot-check” monitor. This type of device is used to collect data from multiple patients and may be easily carried by a healthcare worker such as a nurse during rounds. Today a nurse typically brings a monitor on rounds on a rolling stand. On the other hand, because the monitoring unit 10 according to the present invention is small and light, it can be carried in a pocket or in a waist or shoulder sling. Also, because of the wireless link built into the monitoring unit 10 (FIG. 3) the vital signs collected may be relayed to a central monitoring station or a clinical information system. This may also automatically provide individual vital sign flow sheets for the patient for transmission to and storage in the electronic patient record (EPR) or other history data repository.

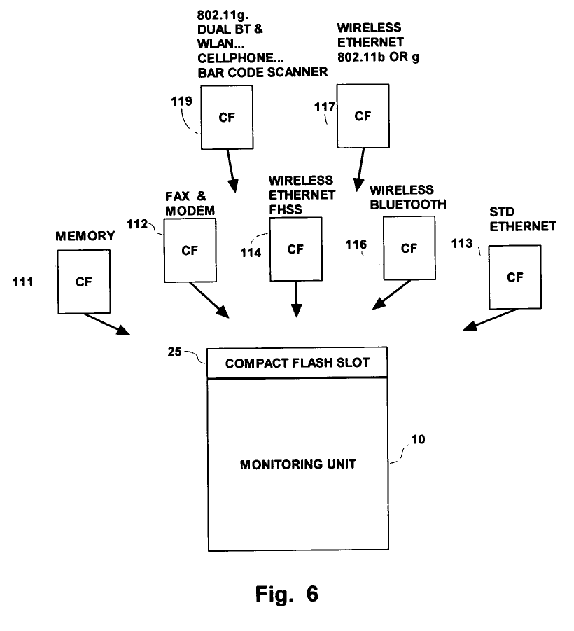

FIG. 6 is a block diagram of the illustrated embodiment shows the monitoring unit 10 equipped with one or more interface ports such as standard “Compact Flash” (CF) slots 25. While expansion pods 71, 72 were described above for interfacing with physiological sensors, the expansion pods 71, 72 may also be selected from a vast array of current storage and I/O modules and any such modules which may be developed in the future. As shown in FIG. 6, the desired modules may selected from: a memory CF card 111, and/or cards providing access to a wide area network (WAN) or local area network (LAN) such as: a fax plus modem CF card 112, a standard Ethernet CF card 113, a wireless Ethernet FHSS CF card 114, a wireless BlueTooth CF card 116, and/or a wireless Ethernet 802.11b and/or g CF card 117. Future cards 119 which may become available for use with the monitoring unit 10 equipped with the compact flash slot 25 include an 802.11g, a dual BT plus WLAN, a cellphone, and/or a barcode scanner CF card, for example. A monitoring unit 10 according to the present invention may operate with any such expansion pods to integrate the functions provided by those expansion pods with those of the monitoring unit 10.

As described above, the monitoring unit 10 is miniaturized and able to operate wirelessly. Because of its small size, it may be used with patients in categories including neonatal, pediatric and adult. The monitoring unit 10 may also employ software modes and algorithms, and be furnished with various physiological limits and constraints, that adapt its operation to those different categories.

The monitoring unit 10 described above is suitable for assignment to and continuous use on one specific patient for the patient's entire length of stay (LOS) in the hospital. When assigned to a patient in this manner, the monitoring unit 10 may support patient monitoring, without requiring removal and reapplication of sensors on the patient, or disconnection and reconnection of any sensor cable connectors, in any of the following clinical situations: (a) an emergency room, (b) an intensive care unit, (c) a pre-operative, intra-operative and post operative environment, (d) ambulatory patient monitoring using wireless telemetry of patient parameter data, (e) hospital ward monitoring and (f) outside the hospital. The monitoring unit 10 is also capable of communicating with a wireless locator system and thereby continuously tracking the location of the patient throughout the hospital.

1. A system for monitoring and processing signal parameters acquired from a patient in multiple operational modes and housed as a portable monitoring unit, comprising:
a data acquisition processor for receiving and processing patient parameter data from a plurality of different patient attached sensors to provide processed patient parameter data;
an image reproduction device for displaying processed patient parameter data;
a communication interface for communicating said processed patient parameter data to:
said image reproduction device for display in a first mode;
a docking station when said portable monitoring unit is docked in said docking station in a second mode; and
a network access point coupled to a communication network via wireless communication in a third mode; and
a power unit for re-charging a battery in said portable monitoring unit in said second mode.

2. A system according to claim 1 wherein:
said communication interface communicates said processed patient parameter data to:
said image reproduction device for display in said first mode;
said docking station when said portable monitoring unit is docked in said docking station in said second mode; and
said network access point coupled to said communication network via wireless communication in said third mode;
without requiring physical removal of said plurality of patient attached sensors.

3. A system according to claim 2 wherein:
said plurality of patient attached sensors are connected to said data acquisition processor through a cable; and
said communication interface communicates said processed patient parameter data to:
said image reproduction device for display in said first mode;
said docking station when said portable monitoring unit is docked in said docking station in said second mode; and
said network access point coupled to said communication network via wireless communication in said third mode;
without requiring physical disconnection of the cable from the data acquisition processor.

4. A system according to claim 3 wherein:
the cable is connected to the data acquisition processor through a connector; and
said communication interface communicates said processed patient parameter data to:
said image reproduction device for display in said first mode;
said docking station when said portable monitoring unit is docked in said docking station in said second mode; and
said network access point coupled to said communication network via wireless communication in said third mode;
without requiring physical disconnection of the connector from the data acquisition processor.

5. A system according to claim 1 wherein said portable monitoring unit is removable from said docking station in said second mode without disconnection of a connector.

6. A system according to claim 1 wherein said portable monitoring unit is removable from said docking station in said second mode without disconnection of a cable.

7. A system according to claim 1 wherein said portable monitoring unit in said third mode supports at least one of, (a) wear by a patient to support monitoring of patient parameters during patient movement and (b) portable use by a healthcare worker to check parameters of multiple patients at different locations.

8. A system according to claim 1 wherein said first and third modes operate concurrently to communicate said processed patient parameter data to said image reproduction device for display and to said network access point coupled to said communication network.

9. A system according to claim 6 wherein said image reproduction device is powered down after a predetermined time interval to conserve power in response to a preprogrammed instruction.

10. A system according to claim 1 wherein in a fourth mode, said communication interface communicates said processed patient parameter data to at least one of, (a) a processor for conditioning said processed patient parameter data for display on a second reproduction device of greater image resolution than said image reproduction device and (b) a processor for conditioning said processed patient parameter data for display on a mobile tablet style reproduction device.

11. A system according to claim 8 wherein said communication interface communicates said processed patient parameter data in said fourth mode by at least one of, (i) wireless and (ii) wired, communication.

12. A system according to claim 1 wherein said processed patient parameter data comprises physiological data including at least one of, (a) electro-cardiograph (ECG) data, (b) blood parameter data, (c) ventilation parameter data, (d) infusion pump related data, (e) invasive or non-invasive blood pressure data, (f) pulse rate data, (g) temperature data and (h) respiratory data.

13. A system according to claim 1 wherein said first, second and third modes support patient monitoring in a plurality of clinical situations including two or more of, (a) an emergency room, (b) an intensive care unit, (c) a pre-operative, intra-operative and post operative environment, (d) ambulatory patient monitoring using wireless telemetry of patient parameter data, (e) hospital ward monitoring and (f) outside the hospital.

14. A system according to claim 1 including an interface port for receiving a compact flash device including at least one of, (a) memory and (b) a card supporting WAN (Wide Area Network) or LAN (Local Area Network) access.

15. A system according to claim 1 wherein said communication interface incorporates a Bluetooth 802.15 compatible wireless transceiver.

16. A system according to claim 1 wherein said communication interface supports network or local communication using wireless technologies including at least one of, (a) WLAN 802.11b standard compatible communication, (b) 802.11a standard compatible communication, (c) 802.11g standard compatible communication, (d) Bluetooth 802.15 standard compatible communication, and (e) GSM/GPRS standard compatible communication.

17. A system according to claim 1 wherein said communication interface automatically switches between wired and wireless operation to maintain continuous communication with at least one of, (a) local point-of-care device, (b) a communication network and (c) a central monitoring station, in response to detection of an operational communication link during a communication link search operation.

18. A system according to claim 1 wherein said portable monitoring unit is assigned to a single particular patient for the duration of the length of stay of said patient in a hospital in multiple hospital care areas.

19. A system according to claim 1 wherein said communication interface communicates with a wireless location detection system and supports patient location tracking.

20. A system according to claim 1 wherein said portable monitoring unit is assignable on-demand to a specific patient to enable a spot-check of vital signs of said patient.

21. A system for monitoring and processing signal parameters acquired from a patient in multiple operational modes and housed as a portable monitoring unit, comprising:
a data acquisition processor for receiving and processing patient parameter data from a plurality of different patient attached sensors to provide processed patient parameter data;
a communication interface for communicating said processed patient parameter data to:
a first docking station when said portable monitoring unit is docked in said first docking station in a first mode;
a network access point coupled to a communication network via wireless communication in a second mode; and
a second docking station when said portable monitoring unit is docked in said second docking station in said first mode;
without requiring physical removal of said plurality of patient attached sensors.

22. A system according to claim 19 wherein:
said plurality of patient attached sensors are connected to said data acquisition processor through a cable; and
said communication interface communicates said processed patient parameter data to:
a first docking station when said portable monitoring unit is docked in said first docking station in a first mode;
a network access point coupled to a communication network via wireless communication in a second mode; and
a second docking station when said portable monitoring unit is docked in said second docking station in said first mode;
without requiring physical disconnection of the cable from the data acquisition processor.

23. A system according to claim 20 wherein:
the cable is connected to the data acquisition processor through a connector; and
said communication interface communicates said processed patient parameter data to:
a first docking station when said portable monitoring unit is docked in said first docking station in a first mode;
a network access point coupled to a communication network via wireless communication in a second mode; and
a second docking station when said portable monitoring unit is docked in said second docking station in said first mode;
without requiring physical disconnection of the connector from the data acquisition processor.

24. A system for monitoring and processing signal parameters acquired from a patient in multiple operational modes and housed as a portable monitoring unit, comprising:
a data acquisition processor for receiving and processing patient parameter data from a plurality of different patient attached sensors to provide processed patient parameter data;
an image reproduction device for displaying processed patient parameter data;
a communication interface for communicating said processed patient parameter data to:
said image reproduction device for display in a first mode;
a docking station when said portable monitoring unit is docked in said docking station in a second mode, said portable monitoring unit being removable from said docking station in said second mode without disconnection of a connector; and
a network access point coupled to a communication network via wireless communication in a third mode; and
a power unit for re-charging a battery in said portable monitoring unit in said second mode.

25. A system for monitoring and processing signal parameters acquired from a patient in multiple operational modes and housed as a portable monitoring unit, comprising:
a data acquisition processor for receiving and processing patient parameter data from a plurality of patient attached sensors to provide processed patient parameter data;
an image reproduction device for displaying processed patient parameter data;
a communication interface for communicating said processed patient parameter data to said image reproduction device for display and for concurrently communicating said processed patient parameter data to:
a docking station when said portable monitoring unit is docked in said docking station in a first mode; and
a network access point coupled to a communication network via wireless communication in a second mode; and
a power unit for re-charging a battery in said portable monitoring unit in said first mode.

26. A system according to claim 22 wherein in a third mode, said communication interface communicates said processed patient parameter data to at least one of, (a) a processor for conditioning said processed patient parameter data for display on a display device of greater image resolution than said image reproduction device and (b) a processor for conditioning said processed patient parameter data for display on a mobile tablet style reproduction device.

27. A method for monitoring and processing signal parameters acquired from a patient in multiple operational modes and housed as a portable monitoring unit, comprising the steps of:
receiving and processing patient parameter data from a plurality of patient attached sensors to provide processed patient parameter data;
communicating said processed patient parameter data to:
an image reproduction device for display in a first mode;
a docking station when said portable monitoring unit is docked in said docking station in a second mode; and
a network access point coupled to a communication network via wireless communication in a third mode;
initiating display of said processed patient parameter data; and
re-charging a battery in said portable monitoring unit in said second mode.
